(19) United States
(12) Patent Application Publication
(10) Pub. No.: US 2019/0070385 A1
(43) Pub. Date: Mar. 7, 2019

(54) SLEEP AID AND BAND FOR SAME

(71) Applicant: Patricia J. Horst, Rockford, IL (US)

(72) Inventor: Patricia J. Horst, Rockford, IL (US)

(21) Appl. No.: 15/696,576

(22) Filed: Sep. 6, 2017

(51) Int. Cl. A61M 21/02; A61B 5/00

(52) U.S. Cl. CPC A61M 21/02; A61M 2021/0027; A61B 5/6831

(57) ABSTRACT

A sleep aid device is provided that pings a user at periodic but diminishing intervals that end after a certain time period that helps facilitate slowing the body down to facilitate sleep. The sleep aid device is preferably worn by the user and most preferably on one's head.

### FIELD OF THE INVENTION

The present invention is generally directed toward sleep aids, and more particularly directed toward sleep aids such as devices that may be worn or used by a person to assist in naturally falling asleep without necessarily using medication.

### BACKGROUND OF INVENTION

One of the common afflictions or problems that affect many persons are sleep problems and sleep disorders in that many people sometimes occasionally or frequently have trouble falling asleep. Various homeopathic and pharmaceutical solutions have presented themselves over the years. However, pharmaceutical solutions are prone to side effects or potentially dependency related issues in some instances. Further, not all homeopathic remedies work for all and there is a constant need for additional improvements and alternative solutions that may be used alone or in conjunction with other devices or methods.

### BRIEF SUMMARY OF THE INVENTION

In an embodiment, the sleep aid and band device can be a simple soft band that may be equipped with a small chip you control that may be worn over the forehead and may cover the eyes or ears of an individual if desired. According to preferred embodiments, the device provides a gentle non-invasive way to send a soft audible repeating tick tone to the individual to help relax and induce sleep that is natural and safe. The device therefore is used for sleep assistance, not for pain, and in preferred embodiments contain no magnetic and no electrical currents that are transmitted into the individual for facilitating the sleep effect (electrical power if used as in an embodiment of an electronic circuit may generate only a soft audible tick tone, and/or if desired some mechanical pressure). The device can periodic ping the user for example by generating a soft audible tick sound that repeats but diminishes in frequency and/or intensity over time.

According to an embodiment, the device naturally trains a person's brain. It can be calibrated to start pinging soft audible “ticks” in a relax mode and can end with a slow-down/fade-out for a natural sleep mode.

According to one inventive aspect, a method of sleep assistance, that comprises: using a sleep aid device; periodic pinging (such as generating a soft audible tick tone, or as otherwise provided) with the sleep aid device; and reducing at least one of a time frequency and a ping intensity of the periodic pinging over time.

In an embodiment, such a method involves physically contacting a user with the sleep aid device during the periodic pinging to physically transmit pinging to the user through contact.

Preferably, the inventive method may involve strapping the sleep aid device to a user with a band.

Preferably, the periodic pinging generates a soft audible tick.

In some embodiments, the sleep aid device comprises an electronic circuit powered by a power source.

Such a method may involve operating the periodic pinging for a time interval of between 5 minutes and 1 hour, and reducing the time frequency over the time interval.

In a more typical example, the periodic ping generates pings at an interval of between 5 and 60 times per minute on average over the first 5 minutes.

The method may involve electrically operating the sleep aid device automatically to generate the periodic pinging with a processer using a power source to electrical power a ping generator device.

The sleep aid device to facility such inventive method may include at least one user input comprising at least one of: time duration input, ping intensity input and ping program input. The method may then further involve selecting such at least one user input to adjust an operational ping parameter of the method.

The method may also involve timing a time period and running a program during the time period to periodically ping and discontinuing the periodic pinging upon expiration of the time period.

In some embodiments, the method reduces the time frequency of the periodic pinging over time.

In some embodiments, the method reduces the ping intensity of the periodic pinging over time.

Another inventive aspect is directed toward a sleep aid device, comprising: a device body having a strap for coupling to a user; a power source; a ping generator; and a processor configured with a program to generate a periodic ping by the ping generator. The program is further configured to reduce at least one of a time frequency and a ping intensity of the periodic pinging over time.

In an embodiment, the strap can be a head band.

The sleep aid device may involve a timer that interacts with the program.

In an embodiment, the sleep aid device comprises an electronic circuit powered by a power source that is adapted to be wireless when the sleep aid device is worn.

In an embodiment, the ping generator is a speaker (e.g. that may be used to generate a soft tick tone).

In an embodiment, the processor is electrical in nature. The ping generator comprises a transducer that converts electrical signals into mechanical motion through a ping generator contact. The ping generator contact is arranged relative to the strap to contact a user when in use.

Other aspects, objectives and advantages of the invention will become more apparent from the following detailed description when taken in conjunction with the accompanying drawings.

### BRIEF DESCRIPTION OF THE DRAWINGS

The accompanying drawings incorporated in and forming a part of the specification illustrate several aspects of the present invention and, together with the description, serve to explain the principles of the invention. In the drawings:

FIG. 1 is a partly schematic illustration of a sleep aid device in the form of an electronic circuit sleep aid device with band mounting and being shown strapped around the forehead of an individual in partly schematic form, in accordance with an embodiment of the present invention;

FIG. 2 is a schematic illustration of an embodiment of an electronic circuit sleep aid device, that may be implemented in such device used in FIG. 1 or other such appropriate device;

FIG. 3 is a schematic process flow diagram of a process and processor used in such processor of an electronic circuit device of the prior embodiments (or alternatively that may be employed in a mechanical processor) in accordance with an embodiment of the present invention;

FIG. 4 is a flow process diagram of an alternative embodiment to that of FIG. 3 and similar in many respects to that of FIG. 3 according to an alternative embodiment;

FIG. 5 is a diagram showing the pinging frequency or intensity of such sleep aid device when in use relative to time;

FIG. 6 is a timeline showing an example of pinging events over the course of several minutes according to an embodiment, understanding that other embodiments may include longer or shorter intervals.

While the invention will be described in connection with certain preferred embodiments, there is no intent to limit it to those embodiments. On the contrary, the intent is to cover all alternatives, modifications and equivalents as included within the spirit and scope of the invention as defined by the appended claims.

### DETAILED DESCRIPTION OF THE INVENTION

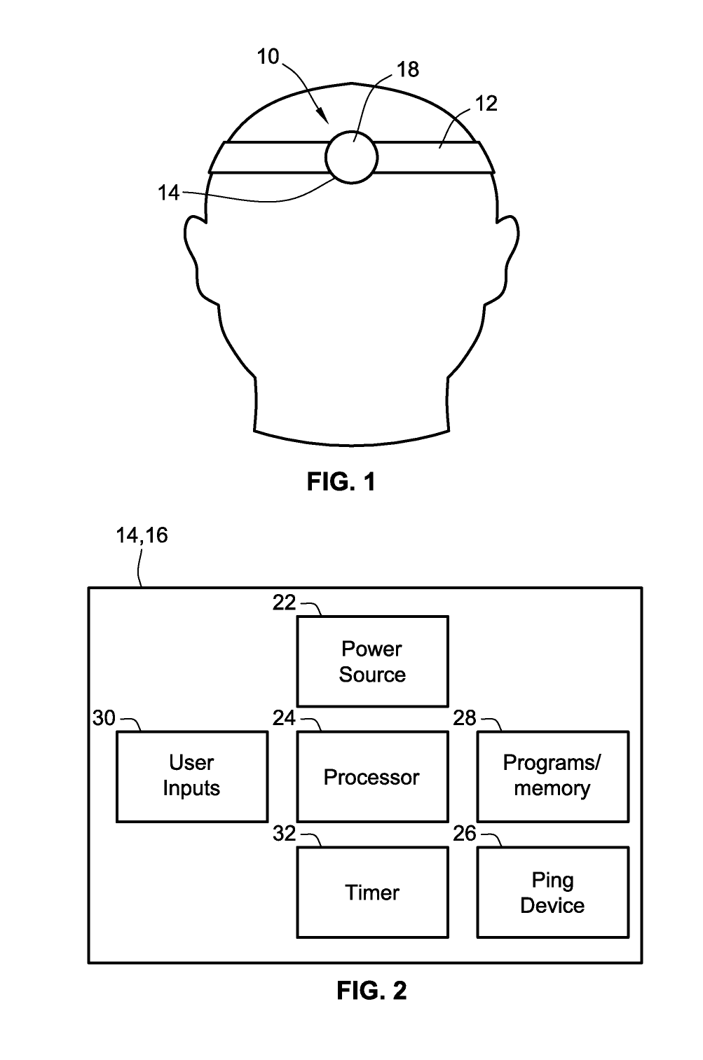

Referring to FIG. 1, a sleep aid device 10 is shown to incorporate a strap which may take the form of a head band 12 as illustrated, that carries and has attached thereto a ping unit 14 that may be an electronic circuit device 16 as shown in FIG. 2. The ping unit typically comprises a case 18 that is carried by the head band 12 that is arranged to be placed in direct contact with a person such as on the forehead 20 of an individual person as shown for example in FIG. 1. It is noted that alternatively, the sleep aid device may be placed at alternative locations on a person such as an arm, but the head location is contemplated to be optimal due to the fact that a person's sensory interactions and brain are located closest to this region. Further, it can be seen that the case 18 in contact with one's forehead is arranged such that it may apply direct “ping” contact with a person which may take the form of a small vibration, such as an applied pressure that is directly and readily felt by an individual sufficiently so that they can sense a slowing down of pinging as will be described herein that is sufficient to help facilitate a sense of slowing down for sleeping. Thus “pinging” as used herein is meant to refer to a periodic signal event that is generated mechanically or electronically.

For example, the pinging may be in the form of a soft audible tick tone such as similar to a clock tick or tone or other audible noise that can provide for such audible tick tone. The tick tone is readily audible, for example, the tone is relatively soft “tick” sound that can be on the order of 0-40 decibels and more preferably 5-30 decibels. As noted herein, the decibel intensity may fade out over time and/or the tick tone frequency.

Accordingly, this sleep aid device can be used to physically contact a user during sleep to facilitate periodic pinging to physically transmitting pinging to the user through contact. While direct contact is preferred as shown in FIG. 1, it is also contemplated that sleep aid device may alternatively be located close to a person with the pinging unit located in close proximity to indirectly provide pinging action to a user in another embodiment.

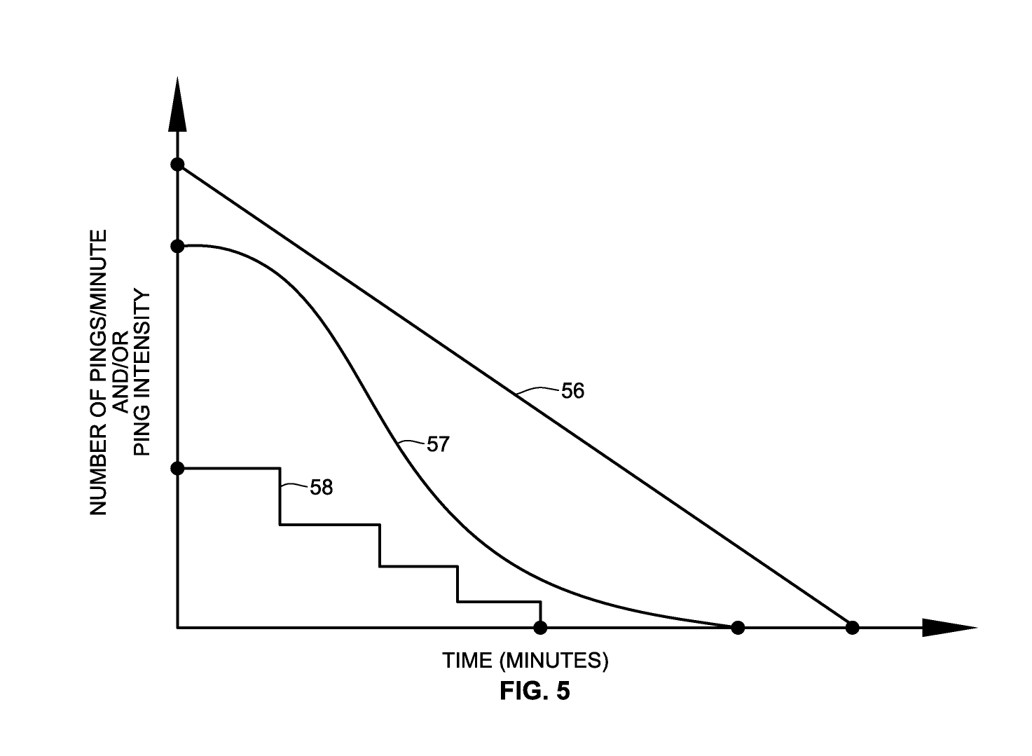

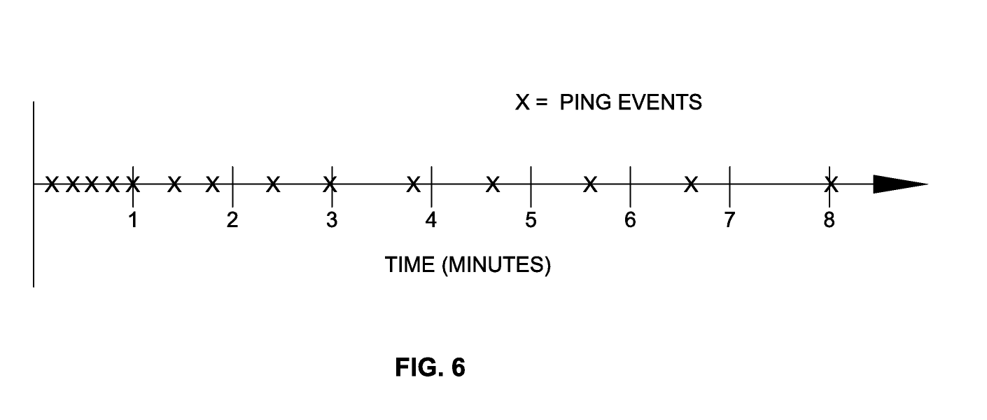

As shown in FIG. 2, a preferred embodiment employs an electronic circuit device 16 powered by a power source 22, that may comprise for example a battery, some sort of capacitor, or even in a less preferred embodiment a cord or wire to an external power supply. In a mechanical embodiment, it may be a loaded spring element. The power source is used to electrically power a processor 24 that is programmed to generate periodic pinging with the sleep aid device via a ping device generator 26 (which may be a speaker, or other transducer). Further, the processor may have pre-programmed programs contained within memory 28 which it can draw from to set a pinging schedule or employ an appropriate algorithm to generate a diminishing ping (e.g. audible tick tone) time frequency or ping (e.g. audible tick tone) intensity such as shown according to the graphs of FIGS. 5 and 6 depicting different possible ping programs that may be employed. Further, user inputs 30 such as a graphic user interface that may be used to do such things as selecting at least one of a time duration input for which pinging may occur, a ping intensity input to control the amount of pressure or audible intensity and a ping program input to determine for example which of the different pinging programs may be desired such as shown with the three alternative programs shown in FIG. 5 as an example. Further, the processor may either integrally include or have associated therewith a timer 32 that can monitor the time over which pinging (e.g. audible tick tone) can occur and be used to identify when pinging may occur.

Standard electrical processors may be used in accomplishing an electronic embodiment and with the program instituted. However it is also contemplated that the processor may be mechanical such as using a mechanical timing wheel with irregular detents that have increased spacing and with the power source being such means as a windable spring. These concepts are used for example in typical mechanical clocks and watches as an example and can also be used here both for facilitate timing and for generating pings such as audible ticks or application of periodic pressure through mechanical motion.

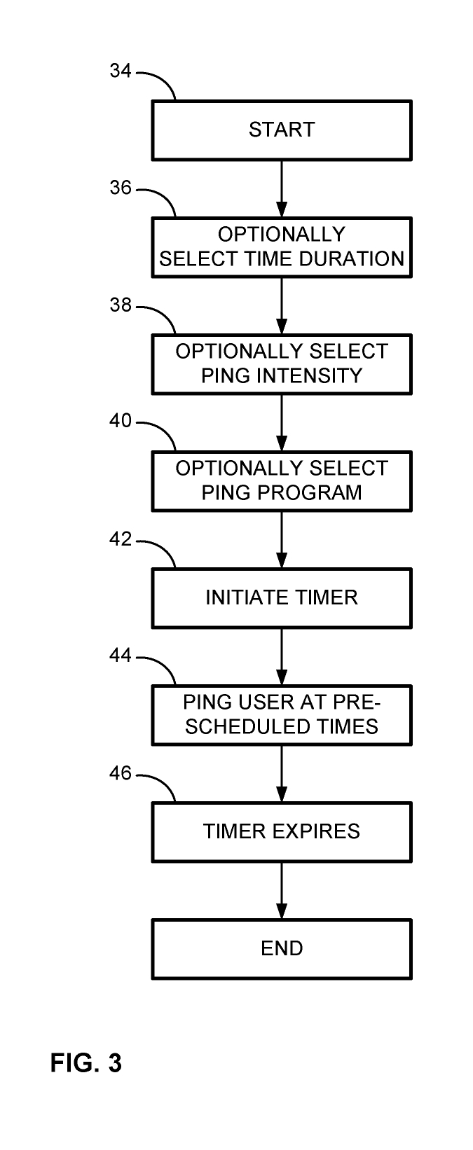

Turning to FIG. 3, one example program is illustrated. When a user wants to use the sleep aid device 10, one initiates the program such as in an electronic timing device by pushing a button (which may be a virtual button if in a graphical user interface or a mechanical button). This provides for the start 34 of the process. In association therewith the user may optionally select time duration 36, may optionally select ping intensity 38 and further may optionally select ping program 40 (such as in the case of pre-programmed ping programs such as shown in FIG. 5). Once the process starts a timer is initiated 42. For example, most typically the time interval for the timer may be between five minutes and one hour which is the duration over which one would typically fall asleep. More typically it is less than an half hour. Thereafter, the processor will ping the user at prescheduled times 44 using the ping generator 20. Once the timer expires 46 meaning that no time remains or that time has expired, the process will end and the pinging is discontinued. At this point it is expected that the user will have fallen asleep. If that is not the case, the user can elect to restart the process again and may also opt to select a different program such as one that has longer pinging intervals as an example considering that more frequent pinging intervals were already used.

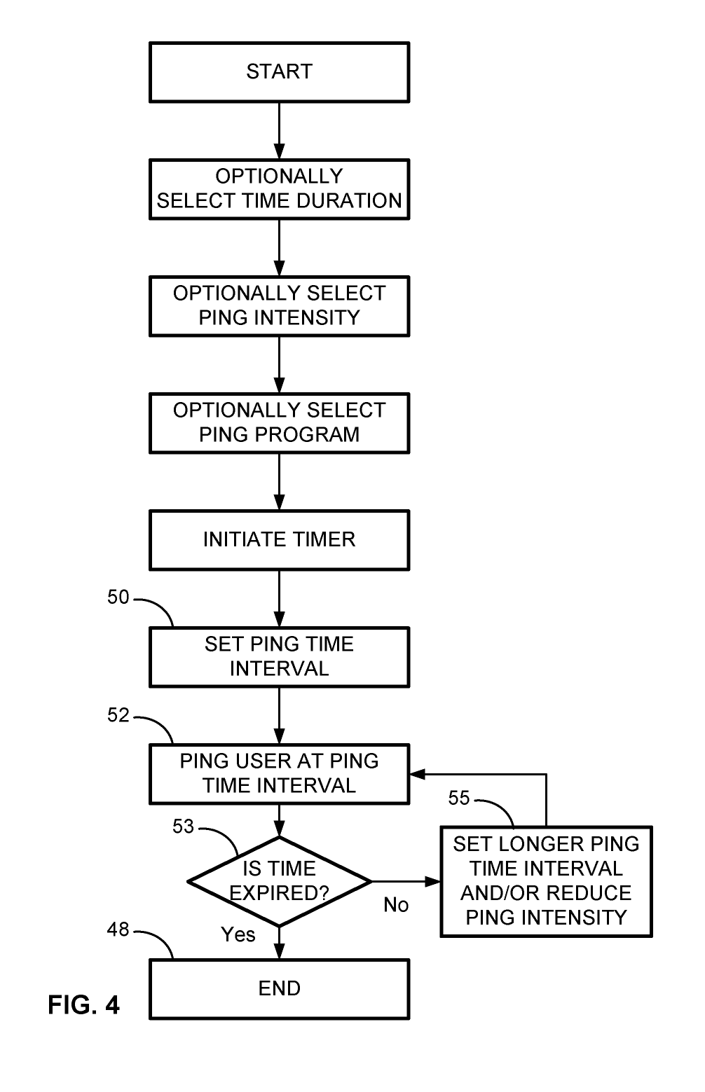

Turning to FIG. 4, a slightly alternative embodiment is shown that contemplates a slightly different logic rather than preset or prescheduled pinging intervals. According to this an algorithm might be used as an example. Only the alternative processes are therefore discussed herein. In FIG. 4 it is shown that alternatively an initial ping time interval may be set 50 and then when that ping time interval is reached by the timer, the processor will ping the user at the ping time interval 52. At that point the processor will ask whether time is expired as shown in processor box 53. If so the process will end 48. If not, it will then set according to process box 55 a longer ping time interval or reduced ping intensity (and then reset to ping the user again at the ping time interval 52 as illustrated).

As shown in FIG. 5, a variety of different ping programs may be used including linear, parabolic type programs that diminish, and stepped type programs. All of these and even those that might temporarily increase pinging frequency intermittently are considered to reduce at least one of a time frequency and ping intensity over periodic pinging over time. Eventually, the pinging is phased out entirely and stops and discontinues. Three different programs 56, 57, 58 showing different diminishing pinging frequency are illustrated in FIG. 5. Again, the program may be preprogrammed with such possibilities or one's own unique programming may be allowed. This may be done either through data lookup tables to establish preset pinging times and/or through algorithms as would be readily understood. Yet another example of an implementation showing pinging over specific time minutes is shown in FIG. 6.

All references, including publications, patent applications, and patents cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) is to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A method of sleep assistance, comprising:
using a sleep aid device;
periodic pinging with the sleep aid device; and
reducing at least one of a time frequency and a ping intensity of the periodic pinging over time.

2. The method of sleep assistance of claim 1, further comprising:
physically contacting a user with the sleep aid device during the periodic pinging to physically transmit pinging to the user through contact.

3. The method of claim 2, further comprising strapping the sleep aid device to a user with a band.

4. The method of claim 1, wherein said periodic pinging generates audible tick.

5. The method of sleep assistance of claim 1, wherein the sleep aid device comprises an electronic circuit powered by a power source.

6. The method of claim 1, further comprising operating the periodic pinging for a time interval of between 5 minutes and 1 hour, and reducing the time frequency over the time interval.

7. The method of claim 1, wherein the periodic ping generates pings at an interval of between 5 and 60 times per minute on average over the first 5 minutes.

8. The method of claim 1, further comprising electrically operating the sleep aid device automatically to generate the periodic pinging with a processer using a power source to electrical power a ping generator device.

9. The method of claim 8, wherein the sleep aid device includes at least one user input comprising at least one of: time duration input, ping intensity input and ping program input, and further comprising selecting the at least one user input to adjust an operational ping parameter of the method.

10. The method of claim 8, further comprising timing a time period and running a program during the time period to periodically ping and discontinuing the periodic pinging upon expiration of the time period.

11. The method of claim 1, wherein the method reduces the time frequency of the periodic pinging over time.

12. The method of claim 1, wherein the method reduces the ping intensity of the periodic pinging over time.

13. A sleep aid device, comprising:
a device body having a strap for coupling to a user;
a power source;
a ping generator;
a processor configured with a program to generate a periodic ping by the ping generator wherein the program is further configured to reduce at least one of a time frequency and a ping intensity of the periodic pinging over time.

14. The sleep aid device of claim 13, wherein the strap is a head band.

15. The sleep aid device of claim 13, wherein a timer interacts with the program.

16. The sleep aid device of claim 13, wherein the sleep aid device comprises an electronic circuit powered by a power source that is adapted to be wireless when the sleep aid device is worn.

17. The sleep aid device of claim 13, wherein the ping generator is a speaker.

18. The sleep aid device of claim 13, wherein the processor is electrical, and wherein the ping generator comprises a transducer that converts electrical signals into mechanical motion through a ping generator contact, the ping generator contact arranged relative to the strap to contact a user when in use.
